(19) United States
(12) Patent Application Publication
(10) Pub. No.: US 2024/0138951 A1
(43) Pub. Date: May 2, 2024

(54) SURGICAL INSTRUMENT SUITABLE FOR PROTECTING TISSUE DURING BONE SURGERY, FOR EXAMPLE WHEN CUTTING OR DRILLING A BONE

(71) Applicant: NEWCLIP INTERNATIONAL, LUXEMBOURG (LU)

(72) Inventors: Jean-Pierre PODGORSKI, SEVREMOINE (FR); Grégoire LARCHE, CHOLET (FR); Marie-Cécile GUILLIN, VERTOU (FR)

(21) Appl. No.: 18/548,474

(22) PCT Filed: Mar. 2, 2022
(86) PCT No.: PCT/EP2022/055294

§ 371 Date: Aug. 30, 2023

(30) Foreign Application Priority Data
Mar. 3, 2021 (FR) 2102061

(51) Int. Cl. A61B 90/00; A61B 17/17

(52) U.S. Cl. CPC A61B 90/08; A61B 17/17; A61B 2017/0092; A61B 2090/08021

(57) ABSTRACT

Disclosed is a surgical instrument suitable for protecting tissue during bone surgery, for example when cutting and/or drilling a bone. The surgical instrument includes a protective portion intended to be positioned in a protective subcutaneous position between the tissue to be protected and a bone. The surgical instrument further includes a temporary stabilization portion which is designed to temporarily secure the surgical instrument to the bone, in the protective position.

Surgical instrument suitable for protecting tissue during bone surgery, for example when cutting or drilling a bone

### TECHNICAL FIELD OF THE INVENTION

The present invention relates to the technical field of surgical instruments which are suitable for protecting tissue during bone surgery, for example when cutting and/or drilling a bone.

### STATE OF THE ART

Bone surgery is surgery performed by orthopedic surgeons, with the aim of repairing the bones following illnesses or accidents.

It encompasses in particular the osteosynthesis techniques: methods allowing treating fractures or mechanical problems on the skeleton using osteosynthesis implants (screws, plates, pins, etc.) placed internally, on the bone, or externally using external fixators, which aim at fastening together bone fragments for the consolidation.

This type of intervention requires different actions on the bones, for example cutting and/or drilling the bones.

However, these interventions can be sensitive, in particular with regard to the different (soft and vascular) tissues surrounding the intervention area.

In order to reduce the risks of the intervention, it is common to implement a surgical instrument, also called “soft and vascular protective tissue” or “protective surgical instrument”, intended to be positioned subcutaneously, between the bone and the soft/vascular portions to be protected.

However, in practice, these current protective surgical instruments are not entirely satisfactory.

Indeed, before the intervention, the surgeon properly positions the protective surgical instrument.

But the different manipulations are likely to move this protective surgical instrument during the intervention and, as a corollary, to no longer ensure its protective role.

There is consequently a need for a technical solution that would allow an optimal protection throughout the intervention.

### PRESENTATION OF THE INVENTION

In order to overcome the aforementioned drawback of the state of the art, the present invention proposes a surgical instrument, suitable for protecting tissue during bone surgery, for example when cutting and/or drilling a bone.

The surgical instrument comprises:
a protective portion intended to be positioned in a protective subcutaneous position between the tissues to be protected and a bone,
which protective portion includes a front face intended to bear on said bone,
a gripping portion, suitable for gripping said surgical instrument,

And, according to the invention, said surgical instrument includes a temporary stabilisation portion which is designed to temporarily secure said surgical instrument to said bone, in said protective position.

Thus, in practice, the surgeon can suitably position the surgical instrument according to the invention to ensure an optimal protection of the different (soft and vascular) tissues surrounding the intervention area.

This positioning is stabilised by the implementation of the temporary stabilisation portion which ensures the temporary securing of this surgical instrument to the bone.

The surgeon can implement different manipulations, without the risk of moving this surgical instrument during the intervention.

The surgical instrument can thus ensure its optimal protective role, throughout the intervention.

This surgical instrument can finally be dissociated from the bone, when the time comes.

Other non-limiting and advantageous features of the product in accordance with the invention, taken individually or according all technically possible combinations, are the following:
the temporary stabilisation portion includes at least one through orifice which is intended to receive a temporarily implantable organ in said bone, for example a pin or a screw, which at least one through orifice is advantageously formed within the protective portion, on the side of the gripping portion, advantageously opening through the front face thereof; preferably, the protective portion has a longitudinal axis, the temporary securing portion includes at least one through orifice which is implanted on one side of said longitudinal axis, which at least one through orifice is advantageously fixed, or movable, over the length of the longitudinal axis of the protective portion, preferably at least one pair of through orifices, implanted on either side of said longitudinal axis, preferably at least two pairs of through orifices, implanted on either side of said longitudinal axis and distributed over the length of said longitudinal axis; more preferably, the temporary securing portion includes at least two non-parallel through orifices, which are advantageously divergent or convergent, each suitable for receiving a pin;
the protective portion is made of a radio-transparent plastic material;
the protective portion includes a groove made of a metal material, opening on the side of said front face and defining a longitudinal axis of said protective portion;
said surgical instrument includes guide means for bone surgery, advantageously means for guiding the cutting of the bone and/or means for guiding the implantation of at least one pin, which guide means for bone surgery are advantageously formed between the protective portion and the gripping portion, and which guide means for bone surgery are advantageously made in one piece or directly mounted; preferably, the means for guiding the implantation of at least one pin comprise a proximal orifice defining a proximal axis extending parallel to a longitudinal axis of the protective portion, directly underlying a cut line intended to be made in said bone, to position a pin involved in guiding the cut line, and/or a distal orifice which defines a distal axis intersecting a cut line intended to be made in said bone, to position at least one pin selected from a cutting stop pin (at the desired bone hinge point) and a pin for guiding a second cut line; even more preferably, the distal orifice is carried by assembly means enabling a mobility over a circular arc travel, advantageously relative to the longitudinal axis of the protective portion and whose centre advantageously coincides with the desired hinge point (advantageously corresponding to the end of the groove), for example a slider which carries said distal orifice and which is carried by a slide guide; most preferably, the means for guiding the implantation are carried by rotating assembly means, enabling a pivotal movement between the protective portion and the means for guiding the implantation (including the proximal orifice) along an axis of rotation parallel to this proximal orifice;
said surgical instrument includes receiving means, suitable for receiving at least one accessory, for example the guide means for bone surgery, advantageously formed between the protective portion and the gripping portion; the receiving means advantageously comprise interlocking means suitable for receiving a complementary base of said accessory.

The present invention also relates to equipment for implementing a bone surgery technique, for example when cutting and/or drilling a bone.

This equipment comprises:
a surgical instrument according to the invention, possibly with guide means for bone surgery,
at least one temporarily implantable organ in said bone, for example a pin or a screw, suitable for temporarily securing said surgical instrument to said bone, in the protective position,
possibly a cutting blade, to make a cut line,
possibly an osteosynthesis plate,
possibly a set of screws for fastening in the bone, intended to be inserted through the through orifices of said osteosynthesis plate to fasten the latter to the surface of the bone.

The present invention also relates to a bone surgery method, which surgical method comprises:
a) a preparation step, during which:
the protective portion of said surgical instrument is positioned between the tissues to be protected and the bone, possibly assisted by radiography,
said surgical instrument is temporarily secured to said bone,
possibly an implantation of at least one pin selected from a pin participating in guiding a cut line (advantageously a single pin participating in guiding a cut line or two pins each participating in guiding a cut line) and a cutting stop pin (at the desired bone hinge point),
b) a bone surgery step,
c) a step of removing the surgical instrument,
d) where appropriate, an osteotomy step, advantageously in the form of a bone opening and a step of fastening an osteosynthesis plate using a set of screws.

Of course, the different features, variants and embodiments of the invention can be associated with each other in various combinations, in so far as they are not incompatible or exclusive with each other.

### DETAILED DESCRIPTION OF THE INVENTION

In addition, various other features of the invention emerge from the appended description made with reference to the drawings which illustrate non-limiting embodiments of the invention and where:

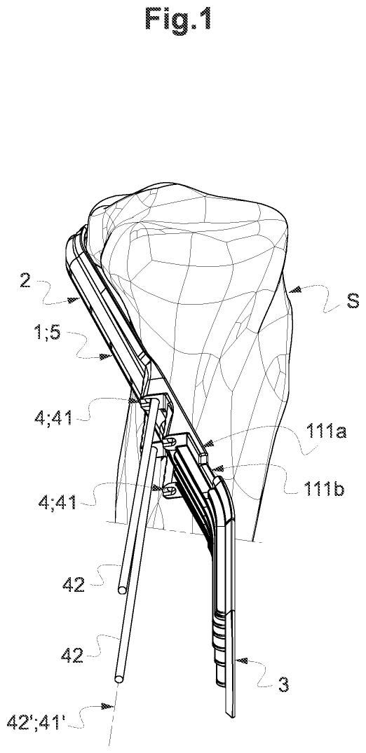

FIG. 1 is a general and perspective view of a surgical instrument according to the invention, comprising a protective part which is temporarily secured to a bone to ensure the protection of tissues (not represented) during bone surgery;

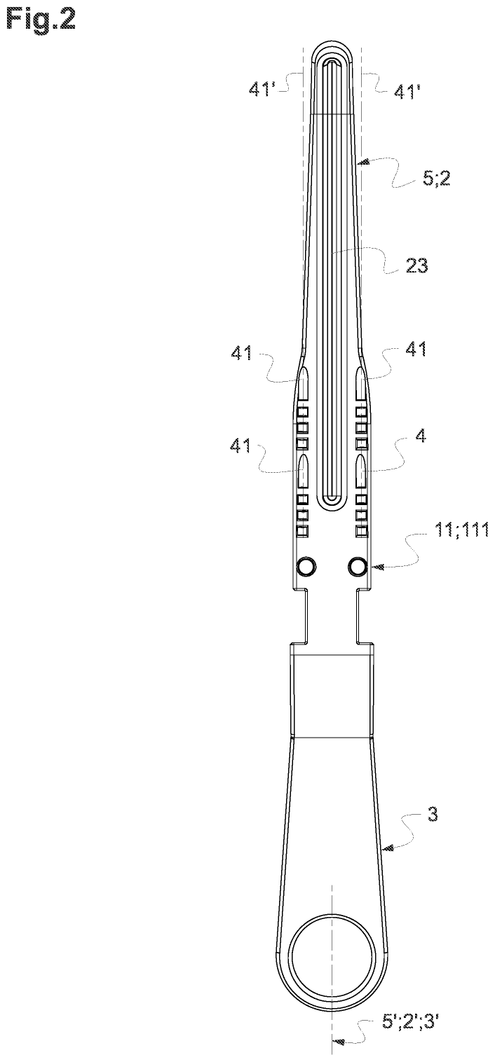

FIG. 2 is a front view of the protective part according to FIG. 1;

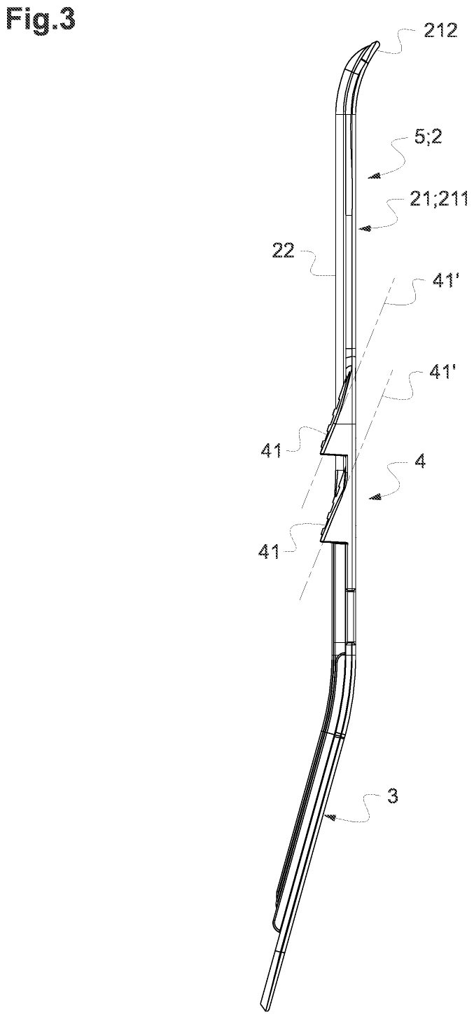

FIG. 3 is a side view of the protective part according to FIG. 1;

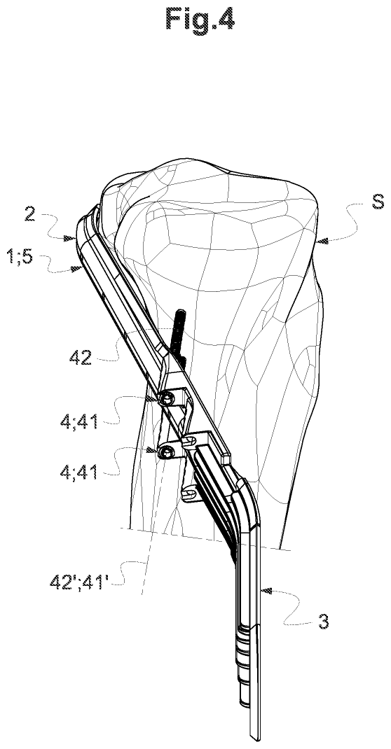

FIG. 4 is a schematic view illustrating a temporary securing of the protective part to the bone by means of screws;

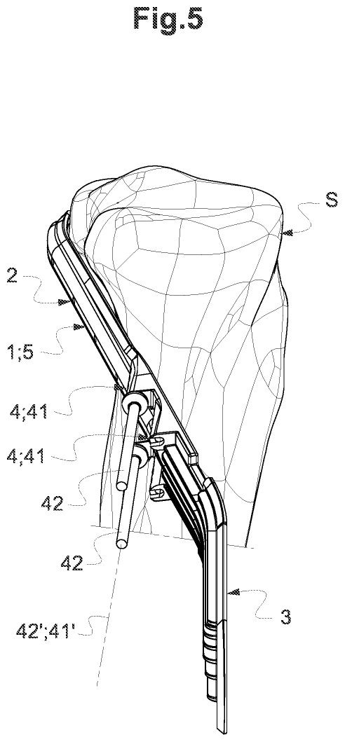

FIG. 5 is a schematic view illustrating a temporary securing of the protective part to the bone using olive pins;

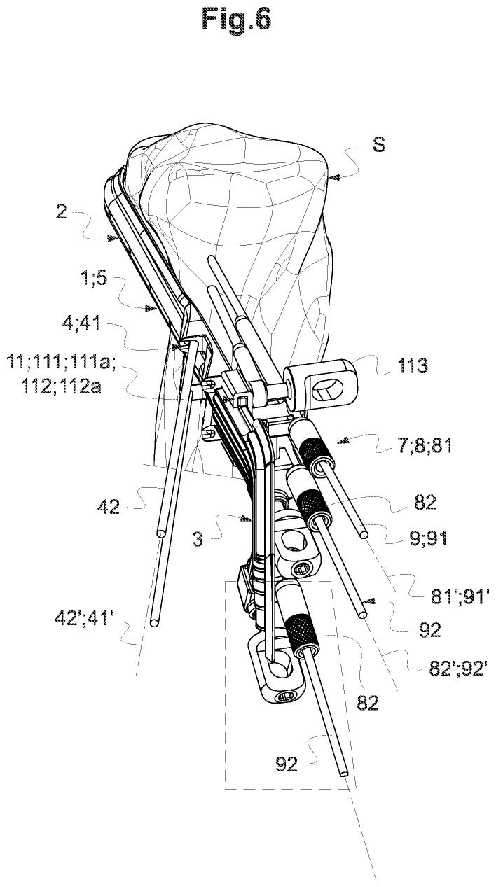

FIG. 6 is a general and perspective view of a surgical instrument according to the invention, comprising a protective part equipped with means for guiding the implantation, temporarily secured to a bone to ensure the protection of tissues during a bone surgery;

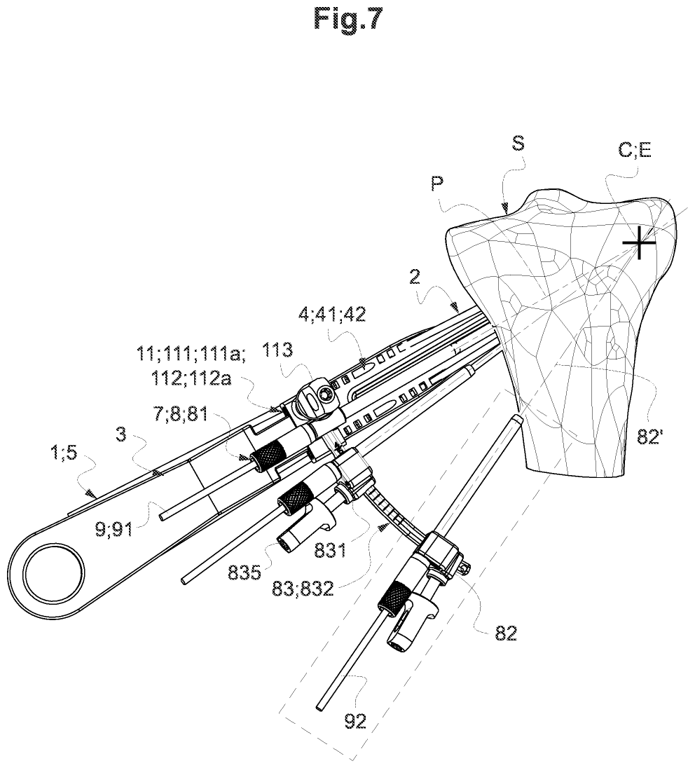

FIG. 7 is a side view of the surgical instrument according to FIG. 6;

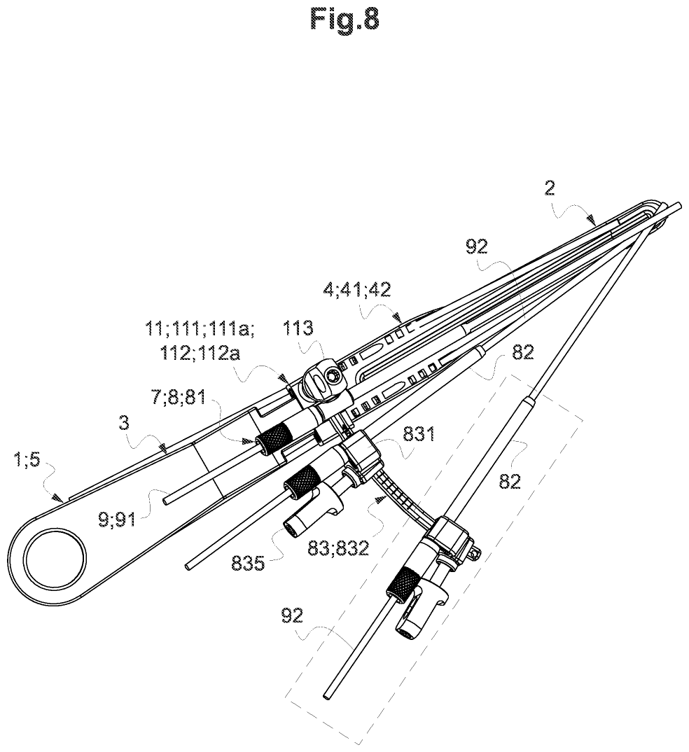

FIG. 8 is a side view showing the surgical instrument according to FIG. 6, in isolation from the bone;

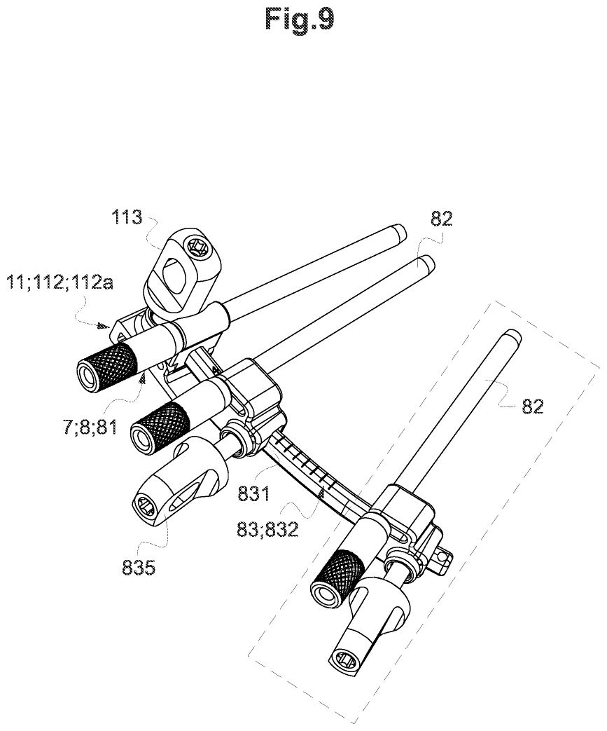

FIG. 9 is a perspective and isolated view of the means for guiding the implantation equipping the protective part according to FIG. 6;

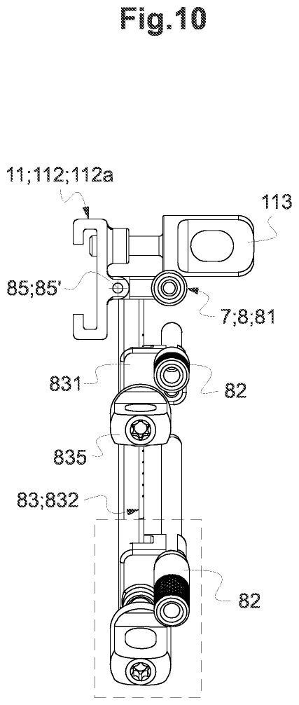

FIG. 10 is an edge view of the means for guiding the implantation according to FIG. 6;

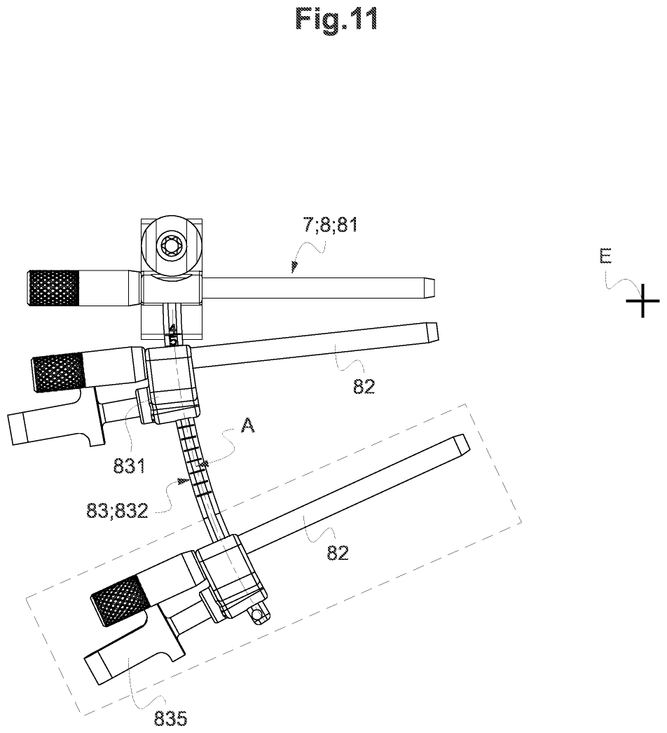

FIG. 11 is a side view of the means for guiding the implantation according to FIG. 6;

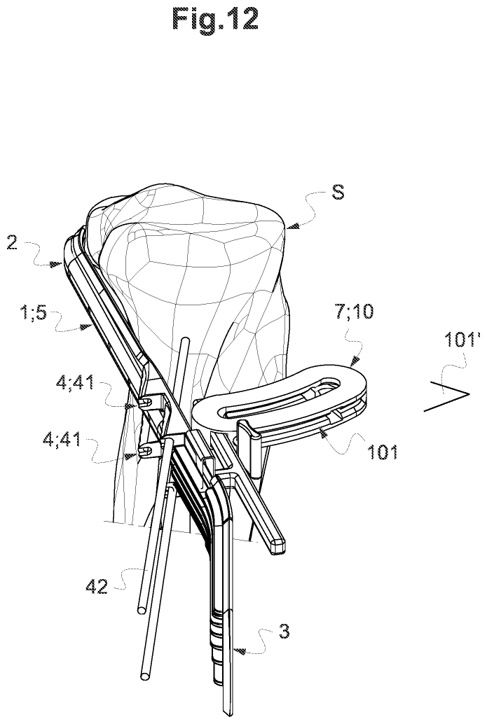

FIG. 12 is a schematic view illustrating the surgical instrument equipped with means for guiding the cutting;

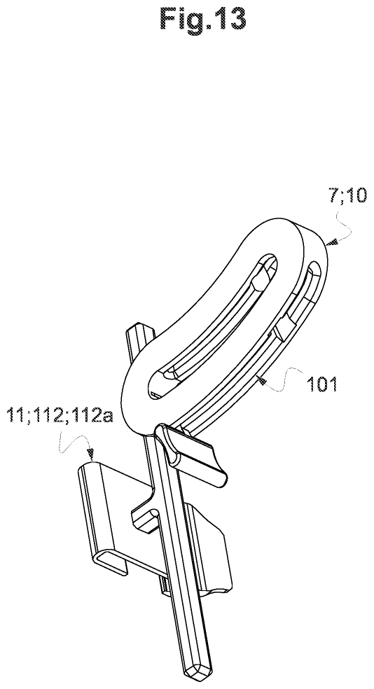

FIG. 13 is a perspective and isolated view showing the means for guiding the cutting;

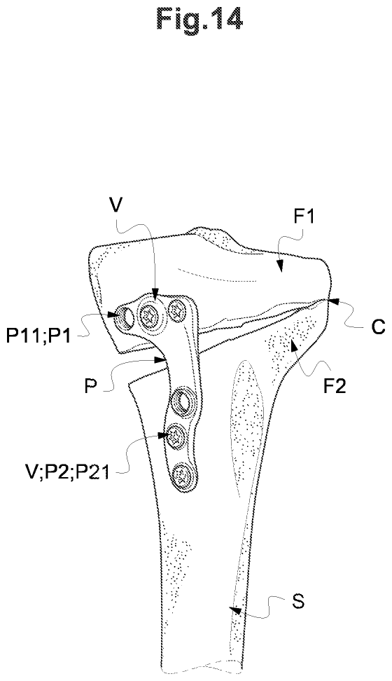

FIG. 14 is a perspective view of the bone equipped with an osteosynthesis plate, following the implementation of the surgical instrument according to the invention.

It should be noted that, in these figures, the structural and/or functional elements common to the different variants can have the same references.

The surgical instrument 1 according to the invention, as illustrated in the figures, is suitable for protecting tissues during a bone surgery, for example when cutting and/or drilling a bone S.

In practice, such a surgical instrument 1, also called “soft and vascular tissue protector” or “surgical instrument” or “ancillary”, is intended to be positioned subcutaneously, between the soft/vascular portions to be protected and the bone S.

This surgical instrument 1 has the role of preserving the soft and vascular portions, when cutting and/or drilling the bone S.

For this and in general, the surgical instrument 1 comprises:
a protective portion 2 intended to be positioned in a protective subcutaneous position between the tissues to be protected (not represented) and a bone S,
a gripping portion 3, suitable for gripping this surgical instrument 1 (advantageously a handle for a manual gripping by the practitioner).

In this case, the protective portion 2 and the gripping portion 3 here are constitutive of a one-piece protective part 5.

The protective part 5 is here in the general form of a spatula, strip or plate.

This protective part 5 advantageously comprises a longitudinal axis 5′, over the length of which the protective portion 2 and the gripping portion 3 are distributed (see in particular FIG. 2).

### Protective Portion

In general, the protective portion 2 is intended to be used as a protective screen between, on the one hand, the tissues to be protected (not represented) and, on the other hand, the bone S on which the surgeon operates.

As illustrated in particular in FIG. 3, the protective portion 2 advantageously comprises two opposite faces:
a front face 21, intended to bear on the bone S and which can be used as a contact surface during cutting/drilling operations, and
an opposite rear face 22 facing the tissues to be protected (not represented).

The protective portion 2 here includes a longitudinal axis 2′, advantageously coaxial with the longitudinal axis 5′ of the protective part 5.

The protective portion 2 advantageously includes a groove 23, which is advantageously rectilinear and advantageously made of a metal material (again forming a longitudinal metal groove).

This groove 23 advantageously consists of a part which is directly mounted on the protective portion 2 and which is made of a metal material.

The groove 23 opens on the side of the front face 21 of the protective portion 2 and here defines the longitudinal axis 2′ of this protective portion 2.

This groove 23 is in particular useful to participate in guiding a cutting blade.

When it is made of a metal material, the groove 23 advantageously has an increased mechanical strength and it can also be used as a visual reference mark when positioning the surgical instrument 1 using an X-ray technique.

The front face 21 of the protective portion 2 advantageously includes:
a main planar portion 211, and
a terminal portion 212, which is curved and protruding from the planar portion 211.

This shape allows the front face 21 to conform to and hook a portion of the circumference of the bone S.

In general, the protective portion 2, or even the protective part 5, is made of a radio-transparent plastic material (with the exception of the groove 23 thereof which is advantageously made of a metal material).

### Gripping Portion

Similarly, the gripping portion 3 here includes a longitudinal axis 3′, advantageously parallel to the longitudinal axis 5′ of the protective part 5.

The gripping portion 3 is here folded relative to the protective portion 2, on the side of the rear face 22 thereof.

### Temporary Stabilisation Portion

According to the invention, the surgical instrument 1 includes a temporary stabilisation portion 4 which is designed to temporarily secure this surgical instrument 1 to the bone, in the protective position (see FIG. 1 in particular).

In this case, the protective portion 2, the gripping portion 3 and the temporary stabilisation portion 4 here are constitutive of the one-piece protective part 5.

The protective portion 2, the gripping portion 3 and the temporary stabilisation portion 4 are advantageously distributed over the length of the longitudinal axis 5′ of the protective part 5.

In this case, the temporary stabilisation portion 4 advantageously includes at least one through orifice 41 which is intended to receive a temporarily implantable organ 42 which is suitable for being temporarily implanted in the bone S.

The temporarily implantable organ 42 thus cooperates with, on the one hand, a through orifice 41 of the temporary stabilisation portion 4 and, on the other hand, the bone S.

The temporarily implantable organ 42 can take different shapes, for example a smooth pin (FIG. 1), a screw (FIG. 4) or even an olive pin (FIG. 5).

The olive pins or the screws can allow a better stability of the surgical instrument 1 in the case where the smooth pins cannot be convergent or divergent.

Such a temporarily implantable organ 42 advantageously includes a longitudinal axis 42′.

Moreover, said at least one through orifice 41 is advantageously formed within the protective portion 2, on the side of the gripping portion 3.

A through orifice 41 advantageously defines a longitudinal axis 41′ which is advantageously intended to define the axis of temporary implantation of the associated temporarily implantable organ 42.

In other words, the longitudinal axis 41′ of a through orifice 41 is advantageously coaxial relative to the longitudinal axis 42′ of the associated temporarily implantable organ 42.

Said at least one through orifice 41 advantageously opens through the front face 21 of the protective portion 2, and advantageously through the rear face 22 of this same protective portion 2.

The longitudinal axis 41′ of a through orifice 41 advantageously extends opposite to the front face 21 of the protective portion 2.

The longitudinal axis 41′ of a through orifice 41 advantageously defines an angle ranging from 5° to 90° relative to the front face 21 of the protective portion 2 (and in particular relative to the planar portion 211 thereof), as illustrated in FIG. 3.

Preferably, said at least one through orifice 41 is implanted on one side of the longitudinal axis 2′ of the protective portion 2. In other words, said at least one through orifice 41 is laterally offset relative to the longitudinal axis 2′ of the protective portion 2 and advantageously relative to the groove 23.

Said at least one through orifice 41 is here fixed along the length of the longitudinal axis 2′ of the protective portion 2. According to an alternative embodiment, which is not represented, said at least one through orifice 41 is movable over the length of said protective portion 2 (for example carried by a carriage which is movable in translation), in a direction parallel to the longitudinal axis 2′ of this protective portion 2.

Preferably, the temporary stabilisation portion 4 includes at least one pair of through orifices 41.

The two through orifices 41 of a pair of through orifices 41 are implanted on either side of the longitudinal axis 2′ of this protective portion 2 (advantageously on either side of the groove 23).

These two through orifices 41 of a pair of through orifices 41 are advantageously disposed transversely relative to the longitudinal axis 2′ of this protective portion 2.

Said at least two through orifices 41 are advantageously non-parallel, preferably divergent or convergent starting from the protective portion 2.

For example, the longitudinal axes 41′ of the through orifices 41 of a pair of through orifices 41 define, therebetween, an angle ranging from 2° to 60° (see FIG. 2).

This “non-parallel” shape allows an optimal stabilisation of the protective portion 2 on the bone S, advantageously without the use of screws.

According to a preferred embodiment, the longitudinal axes 41′ of the two through orifices 41 of a pair of through orifices 41 can extend in the same plane (the longitudinal axis 41′ of these through orifices 41 defines the same angle relative to the front face 21 of the protective portion 2 as illustrated in FIG. 3). And the longitudinal axes 41′ of the two through orifices 41 of a pair of through orifices 41 are advantageously non-parallel in this plane (as illustrated in FIG. 2).

Alternatively, the longitudinal axes 41′ of the two through orifices 41 of a pair of through orifices 41 define different angles relative to the front face 21 of the protective portion 2 (and in particular with respect to the planar portion 211 thereof).

More preferably, the temporary stabilisation portion 4 advantageously includes at least two pairs of through orifices 41 (here two pairs of through orifices 41), distributed over the length of the longitudinal axis 2′ of this protective portion 2.

This embodiment allows adjusting the positioning of the temporary implantation organs 42 on the bone S.

### Guide Means

According to a preferred embodiment, illustrated in FIGS. 6 and following, the surgical instrument 1 incudes guide means 7 for the implementation of bone surgery.

The surgical instrument 1 then advantageously allows a securing of the soft tissues, in a first step, then a cutting aid, in a second time.

The guide means 7 can for example consist of:
means for guiding the implantation 8, suitable for guiding the implantation of at least one pin 9 (FIGS. 6 to 11), and/or
means for guiding the cutting 10, suitable for guiding the cutting of the bone S (FIGS. 12 and 13).

The guide means 7 (for bone surgery) are advantageously formed between the protective portion 2 and the gripping portion 3 (see in particular FIG. 8).

The means for guiding the implantation 8 are described below in relation to FIGS. 6 to 11.

The means for guiding the implantation 8 comprise at least one orifice 81, 82 (advantageously in the form of a tubular conduit or barrel) suitable for receiving and guiding a pin 91, 92, advantageously opposite to the front face 21 of the protective portion 2.

The means for guiding the implantation 8 advantageously comprise a proximal orifice 81 defining a proximal axis 81′ extending parallel to the longitudinal axis 2′ of the protective portion 2.

The proximal orifice 81 is advantageously provided to position a proximal pin 91 participating in guiding a cut line.

The proximal orifice 81, in the form of a tubular conduit, thus guides the proximal pin 91 such that the proximal axis 81′ of the proximal orifice 81 and the longitudinal axis 91′ of the proximal pin 91 are coaxial.

In other words, the proximal axis 81′ advantageously extends parallel to the groove 23 of the protective portion 2 and directly underlying said groove 23.

This proximal orifice 81 is intended to extend directly underlying a cut line intended to be made in the bone S. The proximal orifice 81 is still oriented such that the proximal axis 81′ thereof is parallel to the cut line intended to be made in the bone S.

The cut line is advantageously defined by a plane P passing through the groove 23 and tangent to the proximal pin 91 (FIG. 7).

According to an advantageous embodiment, the means for guiding the implantation 8 are carried by rotating assembly means 85, enabling a pivotal movement between the protective portion 2 and the means for guiding the implantation 8 (including the proximal orifice 81 in particular) along an axis of rotation parallel to this proximal orifice movement between the protective portion 2 and the means for guiding the implantation 8 (including the ‘proximal orifice 81 in particular) along an axis of rotation 85’ parallel to this proximal orifice 81 (see FIG. 10 in particular).

This technical feature is interesting to adapt the cutting plane.

In a complementary or alternative manner, the means for guiding the implantation 8 comprise a distal orifice 82 which is formed at a distance from a cut line intended to be made in the bone S.

This distal orifice 82 defines a distal axis 82′ intersecting a cut line intended to be made in said bone S, to position at least one distal pin 92 selected from a cutting stop pin (at the desired bone hinge point C) and a pin for guiding a second cut line.

The distal orifice 82, in the form of a tubular conduit, thus guides the distal pin 92 such that the distal axis 82′ of this distal orifice 82 and the longitudinal axis 92′ of this distal pin 92 are coaxial.

In order to enable different angulations of the distal pin 92, the distal orifice 82 is advantageously carried by assembly means 83 enabling a mobility over a circular arc travel A (illustrated in particular in FIG. 11).

The distal axis 82′ of the distal orifice 82 thus has an angle which is adjustable relative to the longitudinal axis 2′ of the protective portion 2 (and where appropriate relative to the proximal axis 81′ of the proximal orifice 81).

The centre E of the circular arc travel A is advantageously coincident with the desired hinge point C, also called “opening site” (see FIG. 7).

This centre E of the circular arc travel A advantageously corresponds to the end of the groove 23.

Preferably, the centre E of the circular arc travel A is intended to be located in the space requirement occupied by the bone S, in particular at the opening site.

The centre E of the circular arc travel A allows locating a theoretical point located at a determined distance from the surface of the bone and the plateau (for example, the theoretical point is located 10 to 11 mm from the outer cortical).

For this, the assembly means 83 here comprise a slider 831 which carries the distal orifice 82 and which is itself carried by a slide guide 832.

The slide guide 832 has a circular arc shape, defining the circular arc travel of the slider 831 and of the associated distal orifice 82 thereof.

FIGS. 6 to 11 illustrate the same slider 831, and the distal orifice 82 thereof, in two end-of-travel positions, for the sake of illustration; one of the end-of-travel positions, which is distal, is surrounded in these figures by a dashed line.

The angle of the distal orifice 82 is adjusted depending on the desired functionality for the associated distal pin 92, which is ascending.

As previously mentioned, the distal pin 92 can perform one function of a cutting stop pin (at the desired bone hinge point C) and a pin for guiding a second cut line.

Preferably, the slider 831 is provided with translational locking means 835, suitable for locking the position of the slider 831 over the length of the slide guide 832 after adjustment by the surgeon taking into account the operating diagram.

These translational locking means 835 consist for example of a screw manoeuvred between two positions:
a locking position, in which said screw cooperates with the slide guide 832, and
an unlocking position, in which said screw is spaced apart from the slide guide 832.

Means for guiding the cutting 10 are described in more detail below in relation to FIGS. 12 and 13.

The means for guiding the cutting 10 comprise a slot 101 defining a cutting plane 101′ for the cut line.

The cutting plane 101′ advantageously passes through the longitudinal axis 2′ of this protective portion 2 and advantageously through the groove 23.

Such means for guiding the cutting 10 are for example useful for fully guiding the saw blade in an antero-posterior plane.

In general, the guide means 7 are directly mounted here.

Indeed, the surgical instrument 1 advantageously includes receiving means 11, suitable for receiving at least one accessory, in this case the aforementioned guide means 7 (the accessory and the guide means 7 are designated by the same reference 7 for the sake of simplicity).

The receiving means 11 are advantageously formed between the protective portion 2 and the gripping portion 3, here on the protective part 5.

In this case, the means of reception 11 comprise:
interlocking means 111, formed on the protective part 5, and
a complementary base 112, equipping the accessory 7.

The interlocking means 111 comprise two lateral ribs 111a, advantageously formed on the edge of the protective part 5, and here accessible by two lateral entrails 111b for the assembly/disassembly of the complementary base 112.

The complementary base 112 is then advantageously provided with two complementary grooves 112a, opening opposite to each other (forming a generally C-shaped complementary base) as represented in FIG. 10.

The interlocking of the complementary base 112, on the interlocking means 111, is advantageously obtained by an axial translational movement.

The receiving means 11 advantageously comprise locking means 113 in the assembled configuration.

These locking means 113 consist for example of a screw carried by the complementary base 112 and manoeuvred between two positions:
a locking position, in which said screw cooperates with the interlocking means 111, and
unlocking position, in which said screw is spaced apart from the interlocking means 111.

In an alternative manner, which is not represented, the guide means 7 are made in one piece with at least one other portion of the surgical instrument 1.

In other words, the protective part 5 advantageously includes guide means 7 which are removable or made in one piece.

### Equipment for the Implementation of a Bone Surgery Technique

In general, the present invention also relates to the equipment for the implementation of a bone surgery technique, for example when cutting and/or drilling a bone S.

This equipment, for example in the form of a kit or a set, comprises:
a surgical instrument 1 according to the invention,
at least one temporarily implantable organ 42 in the bone S, for example a pin or a screw, suitable for temporarily securing this surgical instrument 1 with said bone S, in the protective position,
possibly a cutting blade (not represented), to make a cut line in the bone,
possibly an osteosynthesis plate P (FIG. 14),
possibly a set of screws V for fastening in the bone S, intended to be inserted through the through orifices P11, P21 of the osteosynthesis plate P to fix the latter to the surface of the bone S (FIG. 14).

The osteosynthesis plate P is intended to be secured with two bone fragments F1, F2, following the osteotomy technique, so as to freeze the correction made by the surgeon.

Such an osteosynthesis plate P is for example described in detail in the document FR2980967.

Without being limiting, this osteosynthesis plate P is composed of two elongated portions P1, P2, which are each intended to be secured to one of the two bone fragments F1, F2.

The osteosynthesis plate P, here presenting a general L-shape, thus comprises:
a first portion P1 (here proximal portion) intended to be positioned against a first fragment F1 (here proximal fragment) of bone S, and
a second portion P2 (here distal portion) intended to be positioned against a second fragment F2 (here distal fragment) of this same bone S.

Each of these two portions P1, P2 (first/second portion) is provided with a group of through orifices P11, P21 for receiving fastening screws V in one of the bone fragments F1, F2.

### Bone Surgery Method

The present invention also relates to the bone surgery method which comprises:
a) a preparation stage, during which:
the protective portion 2 of the surgical instrument 1 is positioned between the tissues to be protected (not represented) and the bone S, possibly assisted by radiography,
the surgical instrument 1 is temporarily secured to the bone S, by the implementation of the temporary stabilisation portion 4, where appropriate by the implantation of said at least one temporarily implantable organ 42,
possibly an implantation of at least one pin 9, selected from a proximal pin 91 participating in guiding a cut line and/or an aforementioned distal pin 92 (for example selected from a cutting stop pin and a pin for guiding the second cut line),
b) a bone surgery step, for example the cutting and/or the drilling of the bone, preferably a step of cutting the bone S in order to form a bone hinge C connecting two bone fragments F1, F2 (FIG. 14),
c) a step of removing the surgical instrument 1, by inactivating the temporary stabilisation portion 4, where appropriate by removing said at least one temporarily implantable organ 42,
d) where appropriate, an osteotomy step, in this case in the form of a bone opening and a step of fastening an osteosynthesis plate P using a set of screws V (FIG. 14).

The bone surgery step and/or the osteotomy step are advantageously assisted by the aforementioned proximal pin 91 and/or the distal pin 92.

The bone opening movement is carried out by the operator. This opening movement is for example carried out by means of a wedge inserted between the two bone fragments F1 and F2 (or any other appropriate instrument, such as a Mehari forceps).

1. A surgical instrument, suitable for protecting tissue during bone surgery, for example when cutting and/or drilling a bone,
which surgical instrument comprises:
a protective portion intended to be positioned in a protective subcutaneous position between the tissues to be protected and a bone,
which protective portion includes a front face intended to bear on said bone,
a gripping portion, suitable for gripping said surgical instrument,
a temporary stabilization portion which is designed to temporarily secure said surgical instrument to said bone, in said protective position,
which temporary stabilization portion includes at least one through orifice which is intended to receive a temporarily implantable organ in said bone.

2. The surgical instrument according to claim 1, wherein said at least one through orifice is formed within the protective portion, on the side of the gripping portion.

3. The surgical instrument according to claim 2, wherein the protective portion has a longitudinal axis, andwherein the temporary securingstabilization portion includes at least one through orifice which is implanted on one side of said longitudinal axis.

4. The surgical instrument according to claim 3, wherein the temporary stabilization portion includes at least one pair of through orifices, implanted on either side of said longitudinal axis.

5. The surgical instrument according to claim 4, wherein the temporary stabilization portion includes at least two pairs of through orifices, implanted on either side of said longitudinal axis and distributed over the length of said longitudinal axis.

6. The surgical instrument according to claim 2, wherein the temporary securingstabilization portion includes at least two non-parallel through orifices, each suitable for receiving a pin.

7. The surgical instrument according to claim 1, wherein the protective portion is made of a radio-transparent plastic material.

8. The surgical instrument according to claim 1, wherein the protective portion includes a groove made of a metal material, opening on the side of said front face and defining a longitudinal axis of said protective portion.

9. The surgical instrument according to claim 1, wherein said surgical instrument includes guide means for guiding the cutting of the bone.

10. (canceled)

11. (canceled)

12. (canceled)

13. The surgical instrument according to claim 1, wherein said surgical instrument includes receiving means, suitable for receiving at least one accessory, for example the guide means, advantageously-formed between the protective portion and the gripping portion,
which receiving means comprise interlocking means suitable for receiving a complementary base of said accessory.

14. Equipment for implementing a bone surgery technique, for example when cutting and/or drilling a bone, said equipment comprising:
a surgical instrument according to claim 1,
at least one temporarily implantable organ in said bone, for example a pin or a screw, suitable for temporarily securing said surgical instrument to said bone, in the protective position.

15. Bone surgery method comprising:
a) a preparation step, during which:
the protective portion of the surgical instrument according to claim 1 is positioned between the tissues to be protected and the bone, and
said surgical instrument is temporarily secured to said bone,
b) a bone surgery step,
c) a step of removing the surgical instrument.

16. The surgical instrument according to claim 1, wherein said surgical instrument includes guide means for guiding the implantation of at least one pin.

17. The surgical instrument according to claim 16, wherein the means for guiding the implantation comprise a proximal orifice defining a proximal axis extending parallel to a longitudinal axis of the protective portion, directly underlying a cut line intended to be made in said bone, to position a pin involved in guiding the cut line.

18. The surgical instrument according to claim 16, wherein the means for guiding the implantation comprise a distal orifice which defines a distal axis intersecting a cut line intended to be made in said bone, to position at least one pin selected from a cutting stop pin and a pin for guiding a second cut line.

19. The surgical instrument according to claim 17, wherein the means for guiding the implantation comprise a distal orifice which defines a distal axis intersecting a cut line intended to be made in said bone, to position at least one pin selected from a cutting stop pin and a pin for guiding a second cut line.

20. The surgical instrument according to claim 19, wherein the distal orifice is carried by assembly means enabling a mobility over a circular arc travel, relative to the longitudinal axis of the protective portion and whose center coincides with a desired hinge point.
